(19) United States
(12) Patent Application Publication
(10) Pub. No.: US 2018/0333192 A1
(43) Pub. Date: Nov. 22, 2018

(54) APPARATUS AND SYSTEM FOR HEATED ABLATION BALLOON

(71) Applicant: St. Jude Medical, Cardiology Division, Inc., St. Paul, MN (US)

(72) Inventors: John W. Sliwa, San Jose, CA (US); Stephen A. Morse, Menlo Park, CA (US); John P. Goetz, Aptos, CA (US)

(21) Appl. No.: 15/980,422

(22) Filed: May 15, 2018

Related U.S. Application Data
(60) Provisional application No. 62/506,853, filed on May 16, 2017.

(51) Int. Cl. A61B 18/04

(52) U.S. Cl. CPC A61B 18/04; A61B 2018/00577; A61B 2018/046

(57) ABSTRACT

Various embodiments of the present disclosure can include an apparatus. The apparatus can include a balloon and a coil inside the balloon. The apparatus can also include an elongate shaft with a proximal end portion and a distal end portion, where the distal end portion of the shaft is coupled with the balloon. The elongate shaft can include a lumen, wherein the lumen comprises a first lumen portion inside the shaft and a second lumen portion inside the balloon. The apparatus can include a closed loop circulation path where the elongate shaft, the lumen, and the balloon are configured to circulate a fluid through the closed loop circulation path.

### CROSS-REFERENCE TO RELATED APPLICATIONS

This application claims the benefit of U.S. provisional application No. 62/506,853, filed 16 May 2017, which is hereby incorporated by reference as though fully set forth herein.

### BACKGROUND

### a. Field

This disclosure relates to an elongate medical device. In particular, the instant disclosure relates to apparatuses and systems for heated ablation balloons.

### b. Background Art

Electrophysiology catheters are used in a variety of diagnostic and/or therapeutic medical procedures to correct conditions such as atrial arrhythmia, including for example, ectopic atrial tachycardia, atrial fibrillation, and atrial flutter. Arrhythmia can create a variety of dangerous conditions including stasis of blood flow which can lead to a variety of ailments and even death.

Typically in a procedure, a catheter is manipulated through a patient's vasculature to, for example, a patient's heart, and carries one or more electrodes which may be used for mapping, ablation, diagnosis, or other treatments. Once at the intended site, treatment may include radio frequency (RF) ablation, cryoablation, lasers, chemicals, high-intensity focused ultrasound, etc. An ablation catheter imparts such ablative energy to cardiac tissue to create a lesion in the cardiac tissue. This lesion disrupts undesirable electrical pathways and thereby limits or prevents stray electrical signals that lead to arrhythmias. As readily apparent, such treatment requires precise control of the catheter during manipulation to and at the treatment site, which can invariably be a function of a user's skill level.

In the case of an ablation balloon with a heated fluid various tissue can be ablated to create lesions. In the case of ablation balloons heated with radio frequency (RF), maintaining consistent temperatures of a liquid inside the balloon is difficult. A variety of mechanisms and techniques have been employed to heat the fluid in the balloon, but these often require very high power levels and/or require high net fluid flow which can add additional components that increase the cost, size, and complexity of the catheter and system.

### BRIEF SUMMARY

The instant disclosure, in at least one embodiment, an apparatus for providing therapy to tissue comprises a balloon, a coil, where the coil is inside the balloon, a shaft with a proximal end portion and a distal end portion, where the distal end portion of the shaft is coupled with the balloon, and a lumen, where the lumen comprises a first lumen portion inside the shaft and a second lumen portion inside the balloon; and a closed loop circulation path, where the shaft, the lumen, and the balloon are configured to circulate a fluid through the closed loop circulation path.

In another embodiment, a system comprises an elongate medical device with a proximal end and a distal end, wherein the elongate medical device comprises a lumen, a balloon, where the balloon is coupled with the distal end of the elongate medical device and a portion of the lumen is surrounded by the balloon, a coil, where the coil is inside the balloon and is coupled with an exterior surface of the lumen, a pump; and a closed loop circulation path, where the pump circulates a fluid through the closed loop circulation path comprising the balloon and a portion of the lumen.

In yet another embodiment, a balloon therapy apparatus comprises a balloon, a balloon lumen, configured to be coupled with the balloon, with a first opening and a second opening, where the first opening is proximal and configured to be coupled with a distal end of an elongate medical device and the second opening is distal and configured to be coupled with a guide wire, a coil, wherein the coil is coupled with an exterior surface of the lumen and generates heat using an energy source, and a closed loop circulation path, where the balloon lumen includes a first plurality of openings and one or more openings at the proximal end, where the balloon, balloon lumen, and a distal end portion of the elongate medical device are configured to circulate a fluid in the closed loop circulation path.

### BRIEF DESCRIPTION OF THE DRAWINGS

FIG. 1 is an exemplary system diagram of a catheter inserted into a body (the heart), an irrigation system, a computer system with an input/output and display devices.

FIG. 2A is a schematic diagram of a heated ablation balloon apparatus with a closed loop circulation path in an elongate medical device with a balloon, consistent with embodiments in the present disclosure. FIG. 2B is a schematic diagram of a heated ablation balloon apparatus with a closed loop circulation path in an elongate medical device from a handle to a balloon, consistent with embodiments in the present disclosure.

FIG. 2B is a schematic diagram of a heated ablation balloon apparatus with a closed loop circulation path in a proximal portion in an elongate medical device from a handle to a balloon at a distal end portion of an elongate medical device, consistent with embodiments in the present disclosure

FIG. 3 is a schematic view of a heated ablation balloon apparatus that includes an elongate medical device with a balloon coupled to a distal end of the elongate medical device, the balloon including a coil and a balloon lumen with a plurality of openings, consistent with embodiments in the present disclosure.

FIG. 4A is a cross-sectional side view of a heated ablation balloon apparatus that includes an elongate medical device with a balloon coupled to a distal end of the elongate medical device, where the balloon includes a coil and a balloon lumen, consistent with embodiments in the present disclosure.

FIG. 4B is a cross-sectional side view of a heated ablation balloon including an elongate medical device with a balloon coupled to a distal end of the elongate medical device, where the balloon includes a coil and a balloon lumen, consistent with embodiments in the present disclosure.

FIG. 5 is a cross section of the elongate medical device of FIG. 4A along a line 5-5, consistent with embodiments in the present disclosure.

FIG. 6 is a cross section of a portion of the heated ablation balloon apparatus of FIG. 4A, including an inserted guide wire, consistent with embodiments in the present disclosure.

### DETAILED DESCRIPTION OF EMBODIMENTS

The instant disclosure relates to catheters for providing an ablation therapy to tissue, and more specifically, ablation of myocardial tissue with electrophysiology catheters within the heart. In particular, the instant disclosure relates to a balloon that uses energy to heat a fluid inside the balloon. The tissues, such as myocardial tissues including the pulmonary vein, can receive an ablation therapy from the heated fluid within the balloon to alleviate symptoms associated with cardiac arrhythmias. The tissue ablation can produce a consistent tissue ablation line along a length and circumference of the pulmonary venous tissue which substantially blocks the transmission of electrical signals between ectopic foci within the pulmonary vein (PV). Details of the various embodiments of the present disclosure are described below with specific reference to the figures.

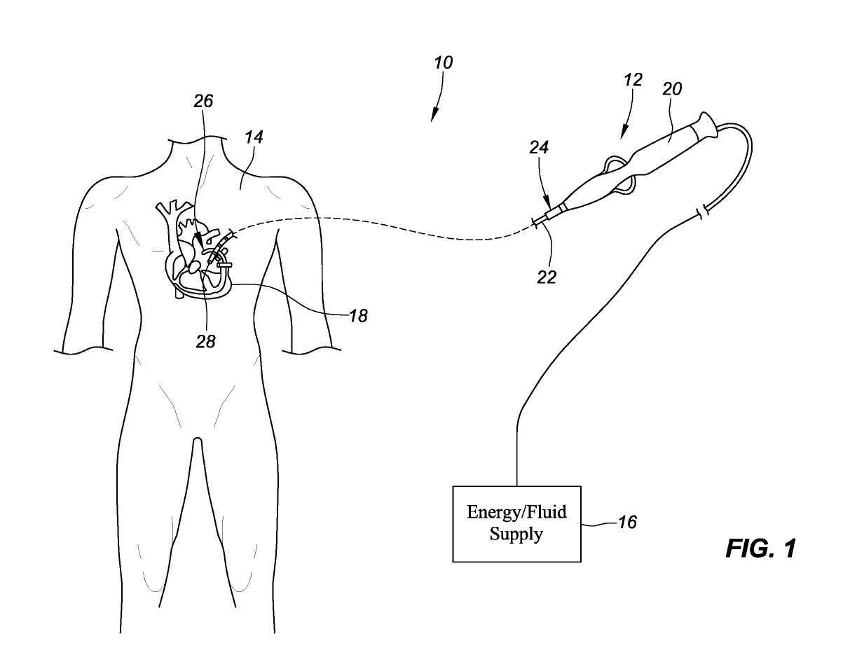

Referring now to the drawings wherein like reference numerals are used to identify similar components in the various views, FIG. 1 is a system diagram showing a medical device and an energy/fluid supply, in accordance with embodiments of the present disclosure. In some embodiments, and with reference to FIG. 1, the system 10 can include a medical device 12 and an energy/fluid supply 16 (e.g., an RF signal generator, a coolant supply, etc.). In an embodiment, the catheter 12 may be an ablation catheter. The catheter 12 can be configured to be inserted into a the patient's heart 18.

The catheter 12 may include a handle 20 and a shaft 22 having a proximal end portion 24, a distal end portion 26, and a tip portion 28 disposed at the distal end portion 26 of the shaft 22. The catheter 12 may further include other conventional components such as, for example and without limitation, a temperature sensor, a position sensor, additional sensors or electrodes, and corresponding conductors or leads. For purposes of illustration and clarity, the description below will be limited to an embodiment wherein the medical device 12 comprises a catheter (a sample catheter is shown in FIG. 1 (e.g., catheter 12)). It will be appreciated, however, that the present disclosure is not meant to be limited to catheters

The shaft 22 can be an elongate, tubular, flexible member configured for movement within the body 14. The tip portion 28 of the shaft 22 supports, for example and without limitation, sensors and/or electrodes mounted thereon. The tip portion 28 may include ablation elements (e.g., ablation tip electrodes for delivering RF energy to fluid in a balloon and/or for delivering RF ablative energy to tissue). The shaft 22 may also permit transport, delivery, and/or removal of fluids (including irrigation fluids, cryogenic ablation fluids, and bodily fluids), medicines, and/or surgical tools or instruments.

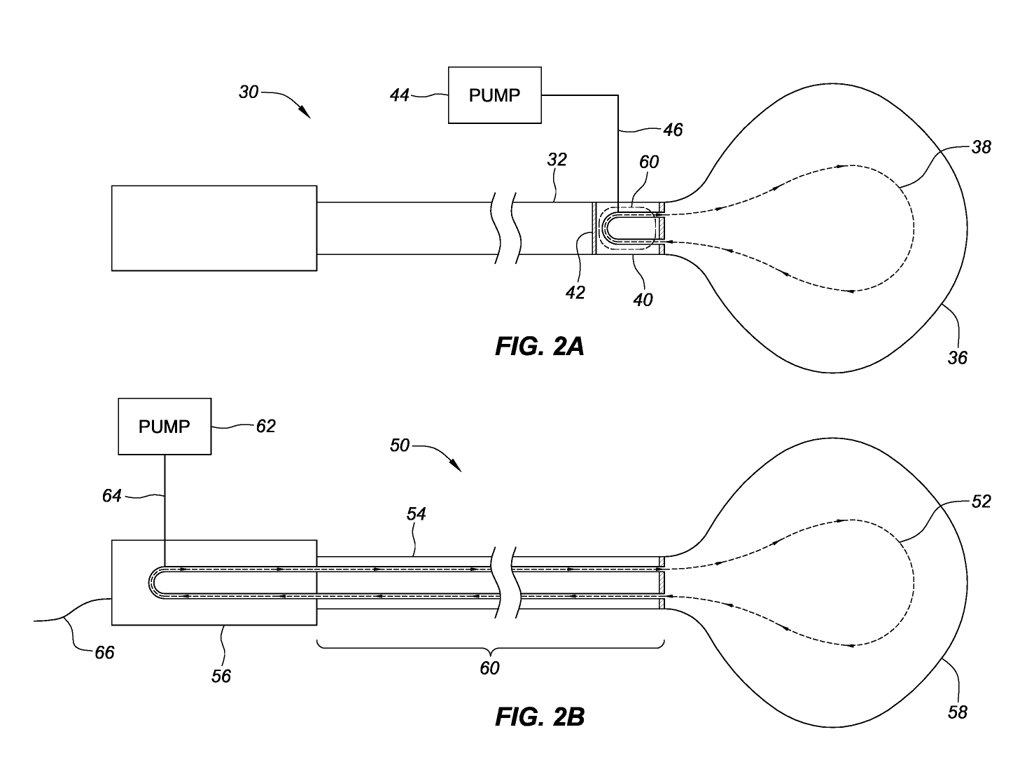

FIG. 2A is a schematic diagram of a heated ablation balloon apparatus with a closed loop circulation path, consistent with embodiments in the present disclosure. The heated ablation balloon apparatus 30 can include the elongate medical device 32 with a distal end 34 coupled to balloon 36 and the closed loop circulation path 38. The closed loop circulation path 38 can be located, for example, at a distal end portion 40 of the elongate medical device 32 and within a portion of the balloon 36. In some embodiments, the closed loop circulation path 38 can be, for example, confined to the distal end portion 40 of the elongate medical device 32 and the balloon 36 by a barrier 42 (e.g., a wall, a divider, a tube, or similar element). The closed loop circulation path 38 can be connected, for example, to a pump 44 by a tube 46. The tube 46 can connect to the distal end portion 40 of the elongate medical device 32 or the balloon 36 (e.g., an example is the distal end portion of the shaft 26 in FIG. 1).

The closed loop circulation path 38 can include any suitable combination of tubes, hoses, openings, orifices, nozzles, fittings and other similar elements that facilitate a flow of a fluid or a gas through the closed loop circulation path 38. In some embodiments, the closed loop circulation path 38 can include a portion of the path through the distal end 40 of the elongate medical device 32. For example, the pump 44 (described in more detail below) can circulate a fluid along the closed loop circulation path 38 from the distal end portion 40 of the elongate medical device 32 into the balloon 36 where the fluid is heated and then flows along the closed loop circulation path 38 out of the balloon 36 back into the distal end portion 40 of the elongate medical device 32, where the closed loop circulation path 38 can recycle back towards the balloon 36.

The closed loop circulation path 38 can include a pump 44. The pump 44 can be any suitable type of pump (e.g., diaphragm, oscillating, reversible peristaltic, etc.) to circulate a fluid or gas. The pump 44 can be connected at any suitable location of the closed loop circulation path 38. For example, the pump 44 can be connected to a portion of the closed loop circulation path 38 near the distal end portion 40 of the elongate medical device 32 or to the balloon 36.

The pump 44 can cause the fluid or the gas to circulate in the closed loop circulation path 38. The circulation of the fluid or the gas can help maintain a more even distribution of a temperature of the fluid or the gas along the closed loop circulation path 38. In some embodiments, the closed loop circulation path can have a low (e.g., zero) net flow amount of the fluid being added and/or removed as the fluid is being heated and then reheated (instead of continually heating new fluid being introduced to the system as used in prior systems). Various sensors can be used to monitor characteristics of the closed loop circulation path 38. For example, characteristics such as the temperature (e.g., a thermocouple), a pressure, and/or a flow rate at one or more locations along the closed loop circulation path 38. The pump 44 and the sensors can be connected to a controller (e.g., an electronic control unit) or similar device to, for example, control and/or monitor the pump 44 and the sensors.

As described above, the pump 44 can be any suitable type of pump. The pump 44 can circulate a fluid (or a gas) in various ways. For example, in one embodiment the pump 44 can cause the fluid to traverse the entire closed loop circulation path 38 (e.g., the fluid or gas does a complete loop including the balloon 36 and the distal end portion 40 of the elongate medical device 32). In another embodiment, the pump 44 can circulate the fluid where the fluid traverses a portion of the closed loop circulation path 38. For example, the fluid (or gas) can be moved back and forth where the forward/backward movement is 25% of a length of the closed loop circulation path 38. A circulation pattern could include one or more circulation variations and the variations could be mixed intermittently. For example, The pump 44 can circulate the fluid in one complete loop of the closed loop circulation path 38, then five partial circulation cycles, then one complete loop, then ten partial circulation cycles, etc.). The pump 44 can also vary the direction (one cycle clockwise then one cycle counter-clockwise, etc.). The circulation can include variations and/or combinations of changes in a direction, a length of time of pump cycles, pumping oscillations, etc.

The elongate medical device 32 can be an elongated, tubular, and flexible member configured for movement within a patient's body 14. The elongate medical device 32 can be coupled with balloon 36 at a distal end 34. The elongate medical device 32 may also permit transport, delivery and/or removal of fluids (including irrigation fluids, cryogenic ablation fluids, and body fluids), medicines, and/or surgical tools or instruments. The elongate medical device 32, which may be made from conventional materials used for catheters, such as polyurethane, can include one or more lumens configured to house and/or transport electrical conductors, fluids, and/or surgical tools. The elongate medical device 32 may be introduced into a blood vessel or other structure within the body 14 through, for example, a conventional introducer sheath.

The balloon 36 can be either of a conductive or a nonconductive material and can be either self-erecting or mechanically erected, such as through the use of an internal balloon. In some embodiments, a balloon and an elongate medical device can combined into a single element (not shown). The balloon 36 can be made of any suitable material (e.g., flexible polymers such as nylon, polyimide (e.g., PEBAX), polyurethane, polyethylene terephthalate (PET), etc.). The balloon 36 can be any suitable shape (round, oval, elliptical, etc.) and can be symmetrical or asymmetrical.

In an exemplary embodiment, a lumen extending through a length of a shaft of the elongate medical device 32 can inject a fluid into the balloon 36 which exerts a radial force on the balloon 36 and thereby expands the balloon 36 into an erect configuration. The lumen (e.g., all of the lumen if more than one or a portion of each lumen) can be thermally insulated. The balloon 36 can also be expanded using, for example, a mechanical structure (not shown) that includes a shape memory material (e.g., nitinol) and/or pull wires or other suitable method and hardware.

FIG. 2B is a schematic diagram of a heated ablation balloon apparatus with a closed loop circulation path in a proximal portion in an elongate medical device from a handle to a balloon at a distal end portion of an elongate medical device, consistent with embodiments in the present disclosure. The heated ablation balloon apparatus 50 can include a closed loop circulation path 52 that can be within an elongate medical device 54, including a handle 56, and a balloon 58. Some embodiments can include a portion of the closed loop circulation path 52 passing through a portion of the balloon 58. Additional embodiments can add a lumen portion 60 to the closed loop circulation path 52 and still other embodiments can include the closed loop circulation path passing through a portion of the handle 56. The lumen portion 60 can be thermally insulated to minimize heat loss from the fluid being circulated in the closed loop circulation path 52. Other parts of the closed loop circulation path 52 can also be thermally insulated (e.g., portions of the handle 56, the tube 64, etc.).

The closed loop circulation path 52 can also, for example, circulate through a portion of the balloon 58 (e.g., a therapy area) and a proximal portion of the elongate medical device 54. In some embodiments, and as illustrated, the closed loop circulation path 52 can also include a portion of the handle 56 at a proximal end portion of the catheter.

In some embodiments, the balloon 58 can be coupled with the elongate medical device 54 where the balloon 58 and the elongate medical device 54 are separate elements. In other embodiments, a balloon and an elongate medical device can combined into a single element (not shown). The balloon 58 can be made of any suitable material (e.g., flexible polymers such as nylon, PEBAX, polyurethane, PET, etc.). The balloon 58 can be any suitable shape (round, oval, elliptical, etc.) and can be symmetrical or asymmetrical.

Similar to FIG. 2A above, the closed loop circulation path 52 can include a pump 62. The pump 62 can be any suitable type of pump (e.g., diaphragm, oscillating, reversible peristaltic, etc.) to circulate a fluid or gas. The pump 62 can be connected to any suitable location of the closed loop circulation path 52 by a tube 64. For example, the pump 62 can be connected by the tube 64 to the handle 56 in embodiments, such as FIG. 2B, where the closed loop circulation path 52 includes a portion inside the handle.

The pump 62 can cause the fluid or the gas to circulate in the closed loop circulation path 52. The circulation of the fluid or the gas can help maintain a more even distribution of a temperature of the fluid or the gas along the closed loop circulation path 52. Various sensors can be used to monitor the temperature (e.g., a thermocouple) and/or the flow rate at one or more locations along the closed loop circulation path 52. The pump 62 and the sensors can be connected to a controller or similar device to, for example, control and/or or monitor the pump 62 and the sensors.

The pump 62 can be used to circulate the fluid in the closed loop circulation path 52. As described above, the pump 62 can be any suitable type of pump. The pump 62 can circulate a fluid (or a gas) in various ways. For example, in one embodiment the pump 62 can cause the fluid to traverse the entire closed loop circulation path 52 (e.g., the fluid or gas does a complete loop including the balloon 58, the elongate medical device 54, and a portion of the closed loop circulation path 52). In another embodiment, the pump 62 can circulate the fluid where the fluid traverses a portion of the closed loop circulation path 52. For example, the fluid (or gas) can be moved back and forth where the forward/backward movement is 25% of the loop. A circulation pattern could include one or more circulation variations or cycles and the cycles could be mixed intermittently. For example, The pump 62 can circulate the fluid in one complete loop, then five partial circulation cycles, then one complete loop, then ten partial circulation cycles, etc.). The pump 62 can also vary the direction of the flow of the fluid (e.g., one cycle clockwise then one cycle counter-clockwise). The circulation can include variations and/or combinations of, for example, changes in a direction, a length of time of pump cycles, oscillations of pumping flow rate, pressure changes, or other similar changes to circulation variables.

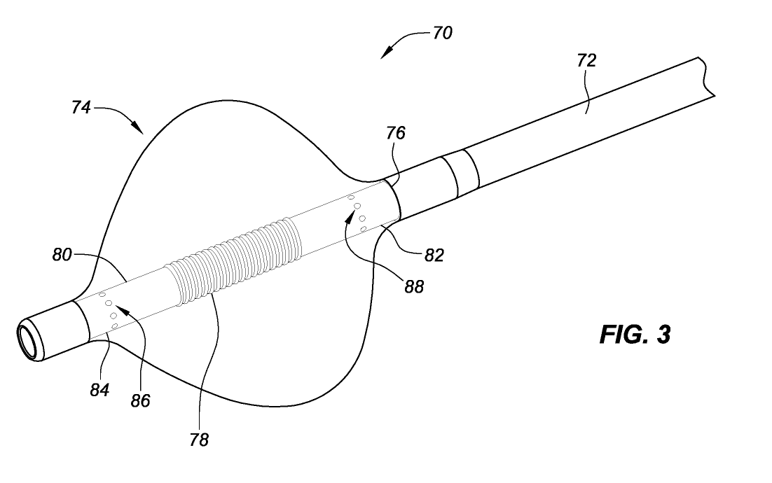

FIG. 3 is a schematic view of a heated ablation balloon apparatus that includes an elongate medical device with a balloon coupled to a distal end of the elongate medical device, the balloon including a coil and a balloon lumen with a plurality of openings, consistent with embodiments in the present disclosure. The heated balloon ablation apparatus 70 can include the elongate medical device 72 which can be coupled with the balloon 74, where the balloon 74 can be coupled to a distal end 76 of the elongate medical device 72, where the balloon 74 includes a coil 78 and a balloon lumen 80.

The balloon lumen 80 can include a balloon lumen proximal end 82 and a balloon lumen distal end 84, with a plurality of openings 86 proximate the balloon lumen distal end 84. In the exemplary embodiment shown in FIG. 3, the plurality of openings 86 are circumferentially distributed about an external surface of the balloon lumen 80. Other configurations are possible with, for example, different spacing of the plurality of openings 86, different sizes of the plurality of openings 86, or other variations. In embodiments described herein, the plurality of openings 86 can be outlets (or inlets) for a fluid provided through the elongate medical device 72.

A pump (e.g., the energy/fluid supply 16 of FIG. 1, the pump 44 of FIG. 2A, or the pump 62 of FIG. 2B) can circulate a fluid to a portion of the elongate medical device 72 and/or a portion of the balloon lumen 80. In one embodiment, a closed loop circulation path can include the fluid flowing out of the plurality of openings 86, circulating through the balloon 74 and then flowing out one or more openings 88 (described in greater detail below).

The balloon lumen 80 can also include one or more openings 88 proximate the balloon lumen proximal end 82. In the exemplary embodiment shown in FIG. 3, the one or more openings 88 are circumferentially distributed about an external surface of the balloon lumen 80. Other configurations are possible with different spacing of the openings, different sizes of openings, etc. In embodiments described herein, the one or more openings at the balloon lumen proximal end 82 can be inlets (or outlets) for a fluid. In some embodiments, the balloon lumen 80 can be a separate element from the elongate medical device 72. In other embodiments, the balloon lumen 80 can be a portion of the elongate medical device 72 (e.g., a single element).

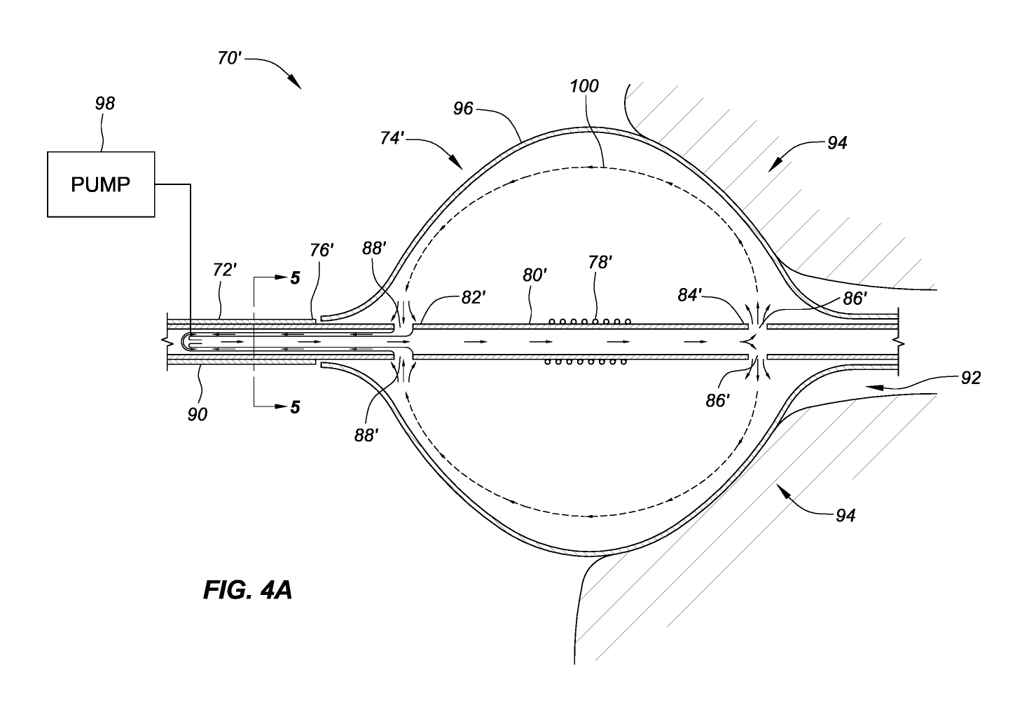

FIG. 4A is a cross-sectional side view of a heated ablation balloon apparatus that includes an elongate medical device with a balloon coupled to a distal end of the elongate medical device, where the balloon includes a coil and a balloon lumen, consistent with embodiments in the present disclosure. The heated ablation balloon apparatus 70′ can include the elongate medical device 72′, and the balloon 74′ coupled to the distal end 76′ of the elongate medical device 72′. The balloon 74′ can include a coil 78′ and a balloon lumen 80′. The balloon lumen 80′ can include a balloon lumen proximal end 82′ and a balloon lumen distal end 84′, with a plurality of openings 86′ at the balloon lumen distal end 84′. In some embodiments, where the balloon 74′ and the elongate medical device 72′ are a single element, the balloon lumen 80′ can include a portion of a lumen that extends through the elongate medical device 72′ and the balloon 74′.

The plurality of openings 86′ can be located at various locations along the balloon lumen 80′. For example, the plurality of openings 86′ can be at a location that is proximate the balloon lumen distal end 84′. In some embodiments, the plurality of openings 86′ can be located alternatively and/or additionally at a location between the balloon lumen proximal end 82′ and the balloon lumen distal end 84′. The plurality of openings 86′ can vary in different ways (e.g., location, number, size, shape, etc.) to allow for variation in treatment of the fluid such as different directions, angles, flow patterns, flow rates, etc. In some embodiments, the plurality of openings 86′ can include, for example, a nozzle, orifice, or other suitable element to control and/or direct the flow of the fluid as it moves through the closed loop circulation path. For example, the nozzle, orifice, or other element can be a separate element or integral to the balloon lumen 80′.

The balloon lumen 80′ can provide fluid, medical devices that include any suitable sensor, wire (e.g., a guide wire), device, and/or other items commonly used in elongate medical device procedures. For example, the balloon lumen 80′ can include a central opening, a hollow tube, and/or a tubular port. In some embodiments, the balloon lumen 80′ can include more than one separate lumens.

The balloon lumen 80′ can also include one or more openings 88′. The one or more openings 88′ can be located, for example, proximate a the balloon lumen proximal end 82′. The one or more openings 88′ can be, for example, spaced radially around a circumference of the balloon lumen 80′ (e.g., similar to the spacing of the plurality of openings 86′) or spaced in other suitable configurations or arrangements. The one or more openings 88′ can be connected to a return tube 90. The return tube 90 can be shaped to direct the fluid coming from the balloon 74′, through the one or more openings 88′, through the return tube 90, then through the balloon lumen 80′, through the plurality of openings 86′, and then back into the balloon 74′ (e.g., together, an embodiment of a closed loop circulation path). In some embodiments, the one or more openings 88′ can each connect to a corresponding return tube 90. In other embodiments, the one or more openings 88′ can connect to a single return tube (e.g., a manifold) that includes or more paths for the fluid to pass into the balloon lumen 80′.

The balloon 74′ can be configured to occlude an opening 92 in a body (e.g., a pulmonary vein in the heart or other opening in the body). When the balloon 74′ is occluding the opening 92, a portion of the balloon 74′ is in contact with tissue 94 (e.g., a therapy site). The portion of the balloon 74′ in contact with the tissue 94 can facilitate therapy of the tissue (e.g., the creation of ablation lesions in, for example, the pulmonary vein (PV) or other locations in the body).

The coil 78′ can be made from any suitable material (e.g., a metal, etc.) and can be coupled with, for example, the balloon lumen 80′. The coil 78′ can be in any suitable configuration regarding the size and configuration of the coil 78′. For example various embodiments of the coil 78′ can have variations in diameter of the wire, variations in the number of wraps of the coil 78′ around the balloon lumen 80′, different spacing of the wraps of the coil 78′, or other configurations. The coil 78′ can be placed at various locations along the length of the balloon lumen 80′. The coil 78′ can be used with RF energy to generate heat (e.g., a heat source). In some embodiments other heat sources can be used. In some embodiments, more than one coil 78′ can be coupled to the balloon lumen 80′ and used for heat generation.

The contents of the balloon 74′ (e.g., a fluid or a gas) can be heated by any suitable means including, for example, RF energy. The fluid can then conduct the heat from the coil 78′ to the balloon 74′ and then to the tissue 94 adjacent the balloon 74′. The fluid can disperse the heat generated by the coil 78′ as the fluid circulates in the balloon 74′ via a closed loop circulation path 100 (discussed in greater detail below). In some embodiments, more than one coil 78′ can be included.

The balloon 74′ can provide heat to the tissue 94 through capacitive heating at a wall 96 of the balloon 74′. The balloon 74′ can include a capacitive substrate (not shown) on either side or both sides of the wall 96 of the balloon 74′. In some embodiments, the capacitive substrate can be heated (e.g., with RF energy), which allows the tissue 94 to be heated (e.g., ablated).

Continuing with FIG. 4A, as the fluid is heated in the balloon 74′, various locations in the balloon 74′ can be different temperatures (e.g., the fluid that is nearer to the sscoil 78′ can be hotter and the fluid further from the coil 78′ and the fluid near the balloon wall 96) can be cooler. A difference in temperature (e.g., a temperature gradient) between the various areas of the balloon can create movement/circulation of the fluid (e.g., a buoyancy gradient) that will cause some distribution of the fluid such as circulation, flow, or movement of the fluid.

A pump (described herein) can circulate the fluid faster and allow for a more uniform distribution of temperatures of the fluid within the balloon. Using the pump with a closed loop to circulate the fluid can be an improved embodiment over systems that are not a closed loop (e.g., an open loop) system and continually pump new coolant into a balloon. In the open loop system additional energy is required to continually heat the new coolant that has replaced the coolant that has been circulated out of the balloon by the pump (e.g., 100% of the coolant that is heated is replaced).

In some embodiments, a pump 98 can circulate a fluid in a closed loop circulation path 100 (similar to examples described in FIGS. 2A-B above). The closed loop circulation path 100 can include, for example, the balloon lumen 80′, the balloon 74′, the return tube 90, the plurality of openings 86′ and the one or more openings 88′.

In some embodiments, the pump 98 can circulate a volume of fluid (e.g., “pulse” or oscillate (“breathe” as in inhaling and exhaling) the volume of the fluid in one direction and then the opposite direction of the closed loop circulation path 100 (e.g., in and out, or clockwise then counter-clockwise, etc.). The pulse of the fluid is not circulated through the entire closed loop and may not be circulated back to the pump. With this embodiment, the fluid is not circulated out of the closed loop circulation path facilitating a higher temperature of the fluid and/or requiring less heat (e.g., RF energy) to maintain a therapy temperature.

In some embodiments, a volume of pulsed fluid can be equal to a volume of a lumen portion that is outside of the balloon (e.g., a length of the lumen with the return tube 90). The lumen portion can be thermally insulated, A stroke of the cycle of the pump can move the volume of the fluid that is equal to the volume of the lumen portion that is outside the balloon and thermally insulated. The oscillation of the fluid that is either within the balloon, and/or the lumen portion that is thermally insulated can reduce heat loss and/or time/energy required to heat the fluid that is circulated into the balloon. This can reduce the amount of reheating necessary to maintain a set temperature of the fluid inside the balloon.

In some embodiments, the volume of incoming (and outgoing) fluid can be within a few percent to maintain a target balloon volume to minimize changes in dimensions of the balloon. This can be considered a partial closed loop circulation system. This embodiment is another example of a low net flow system as new fluid is not continually being added to completely replace the fluid in the system (e.g., inflow rate is 100% and outflow rate is 100%) as used in some prior systems which results in less energy required to maintain a set temperature of the fluid inside the balloon.

A closed loop system with a pump (as described herein) does not require as much energy as the open loop system as the fluid being circulated does not require as much energy to heat since it is being circulated by the pump and heated (e.g., using RF energy) more frequently compared to the open loop. The closed loop system allows for better isothermalization (e.g., the same temperature throughout the balloon from the circulation of the fluid through the pumping action (e.g., oscillating pumping, etc.) of the fluid without requiring a higher net fluid (e.g., water) flow. A high net fluid flow requires higher energy (e.g., RF power) than necessary for therapy (e.g., ablation of tissue) compared to the closed loop system.

In some embodiments, the closed loop circulation path can be modified to be a mostly closed loop circulation path. In this scenario, a small amount of fluid could be added/removed to/from the closed loop circulation path at various intervals (e.g., an amount of time, a volume of fluid circulated, a number of oscillations or pulses, etc.). Adding/removing the small amount of fluid to/from the closed loop circulation system can keep the balloon hotter than other parts of the system the elongate medical device, the lumen, etc.). In embodiments with this arrangement, the heat source (such as a coil using RF energy to heat the fluid) can be operated more frequently and/or with continuous power which can provide more control of the temperature. In some embodiments, without the small amount of fluid being added/removed to the closed loop circulation path, the heat source may be required to cycle on and off more frequently to maintain a desired temperature providing less control of the temperature.

As described above, in some embodiments, the pump 98 can circulate the fluid where the fluid traverses a portion of the closed loop circulation path 100. For example, the fluid (or gas) can be moved back and forth where the forward/backward movement is 25% of the loop. In these embodiments, the plurality of openings 86′ and the one or more openings 88′ can switch roles. For example, (a) the plurality of openings 86′ can output the fluid into the balloon 74′ and the one or more openings 88′ can allow the fluid to exit the balloon 74′ or (b) the one or more openings 88′ can output the fluid into the balloon 74′ and the plurality of openings 86′ can allow the fluid to exit the balloon 74′.

The embodiments described herein can be applied to other balloon structures For example, U.S. Pat. No. 8,052,680, issued on 8 Nov. 2011, and assigned to St. Jude Medical, Atrial Fibrillation Division, Inc. (the '680 patent), includes processes and devices for the treatment of atrial arrhythmia. A catheter and an introducer similar to embodiments described herein could be used with a balloon described in the '680 patent. The '680 patent is hereby incorporated by reference in its entirety as though fully set forth herein.

FIG. 4B is a cross-sectional side view of a heated ablation balloon catheter apparatus including an elongate medical device with a balloon coupled to a distal end of the elongate medical device, where the balloon includes a coil and a balloon lumen, consistent with embodiments in the present disclosure. The heated ablation balloon apparatus 70″ can include the elongate medical device 72″ can include the balloon 74″ coupled to the distal end 76″ of the elongate medical device 72″, where the balloon 74″ can include a coil 78″ and a balloon lumen 80″. The balloon lumen 80″ can include a balloon lumen proximal end 82″ and a balloon lumen distal end 84″, with a plurality of openings 86″ at the balloon lumen distal end 84″ and a plurality of openings 86′″. In some embodiments, where the balloon 74″ and the elongate medical device 72″ are a single element, the balloon lumen 80″ can be a portion of a lumen that includes the elongate medical device 72″ and the balloon 74″.

In the embodiment of FIG. 4B, the plurality of openings 86′″ are shown being proximate the balloon lumen proximal end 82″. In other embodiments the plurality of openings 86′″ can be located at any suitable location between the balloon lumen proximal end 82″ and the balloon lumen distal end 84″. In still further embodiments, additional openings can be located at various locations along the balloon lumen between the balloon lumen proximal end 82″ and the balloon lumen distal end 84″ (e.g., a plurality of openings 86″″, etc.). The additional openings can allow for variations in the flow of the fluid as it circulates in the balloon 74″.

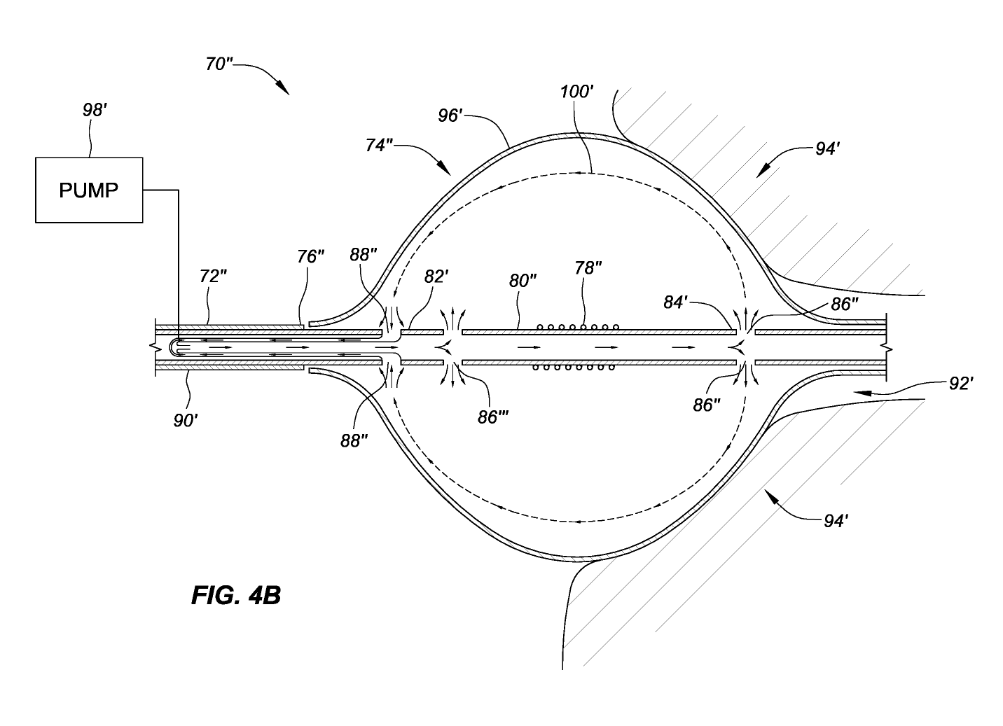

FIG. 5 is a cross section of the elongate medical device of FIG. 4A along a line 5-5, consistent with embodiments in the present disclosure. The cross-sectional view shows an exemplary configuration of the elongate medical device 72′, the balloon lumen 80′, and the return tubes 90′. The lumen interior 102 can be used to supply a fluid to the balloon 74′. The lumen interior 102 can also be used with, for example, a guide wire to maneuver the balloon 74′ to a location in the body 14.

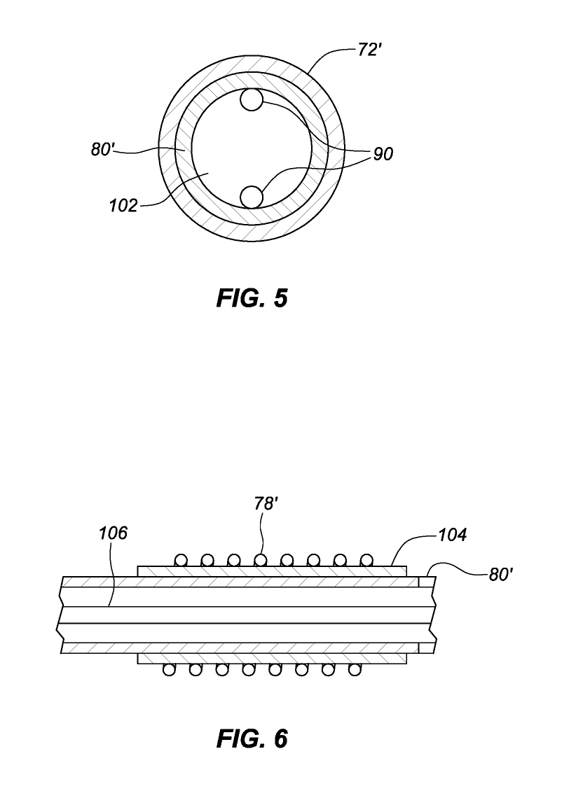

FIG. 6 is a cross section of a portion of the heated ablation balloon apparatus of FIG. 4A, including an inserted guide wire, consistent with embodiments in the present disclosure. FIG. 6 shows additional detail not visible in FIG. 4A. As shown in FIG. 6, the coil 78′ can be wrapped (e.g., helically) around the balloon lumen 80′ with an outer sleeve 104 located between the coil 78′, the balloon lumen 80′, a lumen interior 102 and a guide wire 106. The coil 78′ can include any suitable number of loops. The coil 78′ can be, for example, an RF heating coil. In some embodiments, an outer sleeve 104 can be coupled with the balloon lumen 80′ and the coil 78′. The outer sleeve 104 can be a thermally and electrically insulative material (e.g., polyimide). The electrical insulation of the outer sleeve 104 can prevent the balloon lumen from affecting the generation of RF energy with the coil. The balloon lumen 80′ can be thermally and electrically conductive (e.g., a metal hypodermic tube). The guide wire 106 can be any suitable material (e.g., stainless steel) and can include an insulation layer (e.g., Teflon™).

The guide wire 106 can travel through the lumen interior 102 or, in some embodiments, the guide wire 106 can include a separate lumen (not shown) that is inside the lumen interior 102. The guide wire 106 can be configured to be radially centered in the lumen interior 102 (as shown in FIG. 6) or it can be offset from a central longitudinal axis of the lumen interior 102 (not shown in FIG. 6).

The guide wire 106 can be added to the lumen interior 102 by, for example, a valve (e.g., a hemostasis valve) or similar apparatus located at any suitable location along the elongate medical device and/or the balloon (e.g., balloon 70″). The guide wire 106 can also be inserted through the separate lumen (described above) from any suitable location (e.g., a handle at a proximal end of the elongate medical device).

In some embodiments, the coil 78′ can be coupled with the balloon lumen 80′ without the outer sleeve 104. In embodiments with a guide wire 106, the fluid being circulated through the balloon lumen 80′ can cool the guide wire 106 in the event that the guide wire 106 is not grounded and RF inductive energy couples into the guidewire.

Although at least one embodiment of a heated ablation balloon has been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and can include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure can be made without departing from the spirit of the disclosure as defined in the appended claims.

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An apparatus for providing therapy to tissue, comprising:
a balloon;
a coil, wherein the coil is inside the balloon;
an elongate shaft with a proximal end portion and a distal end portion, wherein the distal end portion of the elongate shaft is coupled with the balloon; and
a lumen, wherein the lumen comprises a first lumen portion inside the elongate shaft and a second lumen portion inside the balloon; and
a closed loop circulation path,
wherein the elongate shaft, the lumen, and the balloon are configured to circulate a fluid through the closed loop circulation path.

2. The elongate medical device of claim 1, wherein the fluid is heated.

3. The elongate medical device of claim 1, wherein the coil generates heat using radio frequency (RF) energy.

4. The elongate medical device of claim 1, wherein the closed loop circulation path includes the balloon, the first lumen portion, and the second lumen portion.

5. The elongate medical device of claim 1, wherein the closed loop circulation path includes the balloon, the first lumen portion, and the second lumen portion, where the second lumen portion goes through a portion of a handle, wherein the handle is connected to a proximal end of the elongate shaft.

6. The elongate medical device of claim 1, wherein the fluid is circulated by a pump.

7. The elongate medical device of claim 6, wherein the pump circulates the fluid in a pattern, wherein the pattern is selected from the group consisting of a one-way pattern, an oscillating pattern, and a variable pattern.

8. The elongate medical device of claim 4, wherein the pattern includes an inflow of a volume and an outflow of the volume in a repeating cycle.

9. The elongate medical device of claim 1, wherein the closed loop circulation path includes an inlet and an outlet to add and to remove a portion of the fluid.

10. The elongate medical device of claim 1, wherein the second lumen portion comprises a plurality of openings at a distal end of the second lumen portion and one or more openings at a proximal portion of the second lumen portion.

11. The elongate medical device of claim 10, wherein the plurality of openings comprises an inlet and the one or more openings comprise an outlet or the plurality of openings comprises an outlet and the one or more openings comprise an inlet.

12. The elongate medical device of claim 2, wherein the closed loop circulation path facilitates even distribution of the heated fluid in the balloon.

13. A system, comprising:
an elongate medical device with a proximal end and a distal end, wherein the elongate medical device comprises a lumen;
a balloon, wherein the balloon is coupled with the distal end of the elongate medical device and a portion of the lumen is surrounded by the balloon and the portion of the lumen comprises an outlet at a distal end of the portion of the lumen and an inlet at a proximal portion of the portion of the lumen;
a coil, wherein the coil is inside the balloon and is coupled with an exterior surface of the lumen;
a pump; and
a closed loop circulation path, wherein the pump circulates a fluid through the closed loop circulation path comprising the balloon and a portion of the lumen.

14. The system of claim 13, wherein the fluid is heated.

15. The system claim 13, wherein the closed loop circulation path further comprises a portion of the closed loop circulation path in a handle, wherein the handle is coupled with the proximal end of the elongate medical device.

16. The system claim 13, wherein the coil generates heat using radio frequency (RF) energy.

17. The system claim 13, wherein the portion of the lumen comprises a plurality of openings at a distal end of the portion of the lumen portion and one or more openings at a proximal portion of the portion of the lumen.

18. The system claim 17, wherein the plurality of openings comprises an inlet and the one or more openings comprise an outlet or the plurality of openings comprises an outlet and the one or more openings comprise an inlet.

19. The system of claim 14, wherein the closed loop circulation path facilitates even distribution of the heated fluid in the balloon.

20. A balloon therapy apparatus, comprising:
a balloon, positioned at a distal end portion of an elongate structure;
a balloon lumen, configured to be coupled with the balloon, with a first opening and a second opening in fluid communication with an interior of the balloon, wherein the second opening is located distally of the first opening;
an electrical coil, wherein the electrical coil is coupled with an exterior surface of the lumen; and
a closed loop circulation path configured to circulate a fluid to and from the balloon and within a portion of the elongate structure.

21. The balloon ablation apparatus of claim 20, wherein the first plurality openings are located at the distal end of the balloon lumen and discharge a fluid from the balloon lumen into the balloon and the one or more openings and discharge the fluid from the balloon.

22. The balloon ablation apparatus of claim 20, wherein the first plurality of openings are located at a proximal portion of the balloon lumen and discharge a fluid from the balloon lumen into the balloon and the one or more openings and discharge the fluid from the balloon.

23. The balloon ablation apparatus of claim 20, wherein the energy source is radio frequency (RF) energy.
